(19) United States
(12) Patent Application Publication
(10) Pub. No.: US 2005/0055099 A1
(43) Pub. Date: Mar. 10, 2005

(54) Flexible spinal disc

(76) Inventor: David N. Ku, Atlanta, GA (US)

Correspondence Address: JONES DAY, 555 WEST FIFTH STREET, SUITE 4600, LOS ANGELES, CA, 90013-1025, US

(21) Appl. No.: 10/658,932

(22) Filed: Sep. 9, 2003

(51) Int. Cl. A61F 2/44

(52) U.S. Cl. 623/17.16

(57) ABSTRACT

A medical device and its use are described. The device is useful for replacement or treatment of a diseased or damaged intervertebral spinal disc. The device has volume to occupy space between vertebral bodies, has mechanical elasticity to provide motion between vertebral bodies, and sufficient strength to withstand the forces and loads on the vertebra. The device may have modifications to allow for attachment to the bones of the vertebrae. The device may also contain modifications for ease of placement in the anatomic space between vertebral bodies. The device may be constructed to expand to restore the normal height of the intervertebral space.

### BACKGROUND OF THE INVENTION

1. Field of the Invention

This invention relates to a prosthetic spinal disc. More particularly, it relates to an implantable artificial spinal disc made of a strong elastomer having the ability to act as a normal disc.

The vertebrate spine is made of bony structures called vertebral bodies that are separated by soft tissue structures called intervertebral discs. The intervertebral disc is commonly referred to as a spinal disc. The spinal disc primarily serves as a mechanical cushion between the vertebral bones, permitting controlled motions between vertebral segments of the axial skeleton. The disc acts as a joint and allows physiologic degrees of flexion, extension, lateral bending, and axial rotation. The disc must have mechanical properties to allow these motions and have sufficient elastic strength to resist the external forces and torsional moments caused by the vertebral bones.

The normal disc is a mixed avascular structure comprised of the two vertebral end plates (“end plates”), annulus fibrosis (“annulus”) and nucleus pulposus (“nucleus”). The end plates are composed of thin cartilage overlying a thin layer of hard, cortical bone that attaches to the spongy cancellous bone of the vertebral body. The end plates act to attach adjacent vertebrae to the disc.

The annulus of the disc is a tough, outer fibrous ring about 10 to 15 millimeters in height and about 15 to 20 millimeters in thickness. The structure of the fibers are like an automobile tire, with 15 to 20 overlapping multiple plies, and inserted into the superior and inferior vertebral bodies at a roughly 30-40 degree angle in both directions. This configuration particularly resists torsion, as about half of the angulated fibers will tighten when the vertebrae rotate in either direction, relative to each other. The laminated plies are less firmly attached to each other. The attached fibers also prevent the disc from extruding laterally with the complex twisting motion of the spine.

Inside the annulus is a gel-like nucleus with high water content. The nucleus acts as a liquid to equalize pressures within the annulus. The material consistency and shape is similar to the inside of a jelly doughnut. The loose fluid-like nature of the nucleus can shrink with compressive forces or swell from osmotic pressure. The ion concentration of the nucleus can create an osmotic swelling pressure of about 0.1 to about 0.3 MPa. As a result, the gel-like nucleus can support an applied load similar to a hydraulic lift. Together, the annulus and nucleus support the spine by flexing with forces produced by the adjacent vertebral bodies during bending, lifting, etc.

The compressive load on the disc changes with posture. When the human body is supine, the compressive load on the third lumbar disc is 300 Newtons (N) which rises to 700 N when an upright stance is assumed. The compressive load increases, yet again, to 1200 N when the body is bent forward by only 20 degrees.

The spinal disc may be displaced or damaged due to trauma or a disease process. A disc herniation occurs when the annulus fibers are weakened or torn and the inner material of the nucleus becomes permanently bulged, distended, or extruded out of its normal, internal annular confines. The mass of a herniated or “slipped” nucleus tissue can compress a spinal nerve, resulting in leg pain, loss of muscle strength and control, even paralysis. Alternatively, with discal degeneration, the nucleus loses its water binding ability and deflates with subsequent loss in disc height. Subsequently, the volume of the nucleus decreases, causing the annulus to buckle in areas where the laminated plies are loosely bonded. As these overlapping plies of the annulus buckle and separate, either circumferential or radial annular tears may occur, potentially resulting in persistent and disabling back pain. Adjacent, ancillary facet joints will also be forced into an overriding position, which may cause additional back pain. The most frequent site of occurrence of a herniated disc is in the lower lumbar region. The cervical spinal disks are also commonly affected.

There are basically three types of treatment currently being used for treating herniated or degenerated discs: conservative care, discectomy and fusion. The majority of patients with low back pain will get better with conservative treatment of bed rest.

Discectomy can provide excellent short-term results. However, a discectomy is not desirable from a long-term biomechanical point of view. Whenever the disc is herniated or removed by surgery, the disc space will narrow and may lose much of its normal stability. The disc height loss may cause osteo-arthritis changes in the facet joints over time. The normal flexibility of the joint is lost, creating higher stresses in adjacent discs. At times, it may be necessary to restore normal disc height after the damaged disc has collapsed.

Fusion is a treatment by which two vertebral bodies are fixed to each other by a rigid piece of metal, often with screws and plates. Current treatment is to maintain disc space by placement of rigid metal devices and bone chips that fuse two vertebral bodies. The devices are similar to mending plates with screws to fix one vertebral body to another one. Alternatively, hollow metal cylinders filled with bone chips can be placed in the intervertebral space to fuse the vertebral bodies together (e.g. LT-Cage™ from Sofamor-Danek or Lumbar I/F CAGE™ from DePuy). These devices have significant disadvantages to the patient in that the bones are fused into a rigid mass with no flexible motion or shock absorption that would normally occur with a natural spinal disc.

Fusion generally does a good job in eliminating symptoms of pain and stabilizes the joint. However, because the fused segment is fixed, the range of motion and forces on the adjoining vertebral discs are increased, possibly enhancing their degenerative processes. Fusions were also done for knee joints, previously; however, this treatment fell out of favor with the advent of movable total knee prostheses.

Some recent devices have attempted to allow for motion between the vertebral bodies through metal and hard plastic devices that allow some relative slippage between parts (e.g. ProDisk, Charite, see, for example, U.S. Pat. Nos. 5,314,477, 4,759,766, 5,401,269 and 5,556,431). The rigid pieces of these devices allow for some relative motion but no shock absorption.

More recently, several prosthetic spinal disc nucleus devices have been proposed. The devices fit in the space of the herniated nucleus and require a constraining jacket or an intact annular ring to hold a liquid-like nuclear prosthesis in a cavity. These devices may extrude, leak, or herniate through the damaged annulus, resulting in significant pain.

Degenerated, painfully disabling interspinal discs are a major economic and social problem. Any significant means to correct these conditions without further destruction or fusion of the disc may serve an important medical role in the treatment of patients. A substantial need exists for an implantable prosthetic spinal disc, which restores the size, load bearing ability, and flexibility of the spinal disc. Further, there is need for a simple prosthesis that will restore disc height in a slow manner after placement. Ideally, the disc height should be restored over a time period greater than 3 hours but less than 3 months.

2. Description of the Prior Art

Artificial spinal discs are known in the prior art. U.S. Pat. No. 4,309,777 to Patil, relates to a prosthetic utilizing metal springs and cups. A spinal implant comprising a rigid solid body having a porous coating on part of its surface is shown in Kenna's U.S. Pat. No. 4,714,469. An intervertebral disc prosthetic consisting of a pair of rigid plugs to replace the degenerated disc is referred to by Kuntz, U.S. Pat. No. 4.349,921. U.S. Pat. No. 3,867,728, to Stubstad et al., relates to a device, which replaces the entire disc made by laminating vertical, horizontal or axial sheets of elastic polymer. U.S. Pat. No. 4,911,718 to Lee et al., relates to an elastomeric disc spacer comprising three different parts; nucleus, annulus and end-plates, of different materials. Lee teaches a disc made of a specific layered structure of 3-24 separated laminas, unidirectional reinforcing fiber, and specific orientation of these components. U.S. Pat. No. 3,875,595 to Froning relates to a collapsible plastic bladder-like prosthetic of nucleus pulposus. U.S. Pat. Nos. 4,772,287, and 4,904,260, by Ray, et al. describe cylindrical prosthetic disc capsules with or without therapeutic agents. U.S. Pat. Nos. 5,674,295, and 5,824,093 to Ray et al. teach nucleus prostheses with a hydrogel core and a constraining jacket that are pillow shaped or capsule shaped. Bao et al., in U.S. Pat. Nos. 5,047,055 and 5,192,326, describe artificial nuclei comprising hydrogels in the form of large pieces shaped to conform to the shape of the disc cavity or beads within a porous envelope, respectively. Another variation of a nucleus replacement is described by Bao et al. in U.S. Pat. No. 5,534,028 for variations in posterior and anterior modulus.

The intervertebral disc is a complex joint anatomically and functionally and it is composed of three component structures, each of which has its own unique structural characteristics. To design and fabricate such a complicated prosthesis from acceptable materials which will mimic the function of the natural disc is very difficult. The new design disclosed here provides the solution to a very difficult problem.

The disadvantage of metal or rigid disc replacements is that they do not provide any shock-absorbing elasticity or flexibility in multiple planes. The Kuntz device uses rigid plugs to replace the disc space. The multiple components required in the previous designs by Stubstad et al. and Lee are difficult to fabricate and install. The Lee devices are too weak as an entire disc replacement, are complex to fabricate, and do not restore disc height over time.

These problems are not solved by Froning and Ray et al., who use bladders, or capsules, respectively, which are filled with a fluid or thixotropic gel. Their devices contain a fluid that must be completely sealed to prevent fluid leakage. These devices have a tendency to leak fluid or extrude with the range of motion associated with normal spine bending and twisting. Ray further requires an inelastic covering. The patents from Bao et al., teach toward a hydrogel prosthetic lumbar disc nucleus that is substantially weaker than an entire disc. This nucleus works by distributing the vertical load to the damaged or repaired natural annulus ring in an effort to prevent the prosthetic nucleus from bulging and herniating.

A further problem is that the prior elastic devices have a tendency to dislodge or extrude from the intervertebral space.

### SUMMARY OF INVENTION

The object of the present invention is to provide a novel spinal disc replacement that is flexible yet strong, can act as a mechanical shock absorber and allow flexibility of motion between the vertebrae. The device is a permanent medical implant for use as a spinal disc. The present invention has a compressive modulus of elasticity that is similar to the normal spinal disc over a range of 0.1 MegaPascals (MPa) to 10 MPa. This is much more compliant than previously used metals or high molecular weight polyethylene plastics with a compressive modulus typically greater than 100 MPa. The elasticity of the present invention allows for shock absorption and flexibility.

The present invention is also novel in that it is made of a solid material that does not leak. The Bao and Ray patents describe a liquid component or a soft jelly component that can leak and extrude.

In general, any elastomer that can be used for biomedical purposes can be used as long as the elastomer exhibits a compressive strength of at least 1 MPa, preferably 10 MPa when subjected to the loads of the human spine. The elastomer should preferably have an ultimate stretch of 15% or greater and an ultimate tensile or compressive strength of 100 kiloPascals or greater. Hydrophilic polymers are preferred for biocompatibility and controlled swelling characteristics.

The present invention further contains modifications for fixation or adhesion that further prevent extrusion of the device. The fixation may be achieved through modification of the cranial and caudal faces of the device to allow fibrous attachment and friction, or the device may have material extensions from the faces or circumference of the device that allow surgical fixation to the vertebral bodies.

Further the prosthesis may swell or expand over time to restore disc height in a controlled manner, and allow fixation in situ. While the Ray devices can be inflated at time of placement, none of the prior art describes a device with controlled swelling properties that passively change size in a physical dimension.

The device acts mechanically as a normal spinal disc, provides for attachment to the endplates of the vertebral bodies, and expands to restore the normal height of the intervertebral space. It is envisioned that this prosthetic spinal disc would be inserted by a surgical procedure into the intervertebral space. It may be used for separation of two bony surfaces within the spine or in other parts of the body. The prosthesis may find use in humans or as a veterinary medicine device.

The shape of the device is a complicated, three-dimensional structure that provides both anatomical shape and mechanical support. The anatomical shape has an irregular volume to fill the intervertebral disc space. The coordinates of the body can be described using the anatomic directions of superior (towards the head), inferior (towards the feet), lateral (towards the side), medial (towards the midline), posterior (towards the back), and anterior (towards the front). From a superior view, the invented device has a kidney shape with the hilum towards the posterior direction. The margins of the device in sagittal section are generally contained within the vertebral column dimensions.

### BRIEF DESCRIPTION OF THE DRAWINGS

FIG. 1 is a perspective view of the invented prosthetic spinal disc.

FIG. 2 is a side, anterior view of the prosthetic spinal disc.

FIG. 3 is a cranial or superior view of the prosthetic spinal disc.

FIG. 4 is a perspective view of a preferred prosthetic spinal disc with extensions for attachment to the vertebral body.

FIG. 5 is a perspective view of a preferred prosthetic spinal disc with fibers or surface treatments on the cranial face.

FIG. 6 is a perspective view of a preferred prosthetic spinal disc.

FIG. 7 is a cranial view of a spinal segment including a degenerated discal area.

FIG. 8 is a side view of a human disc space with a prosthetic spinal disc implanted.

### DETAILED DESCRIPTION OF THE PREFERRED EMBODIMENTS

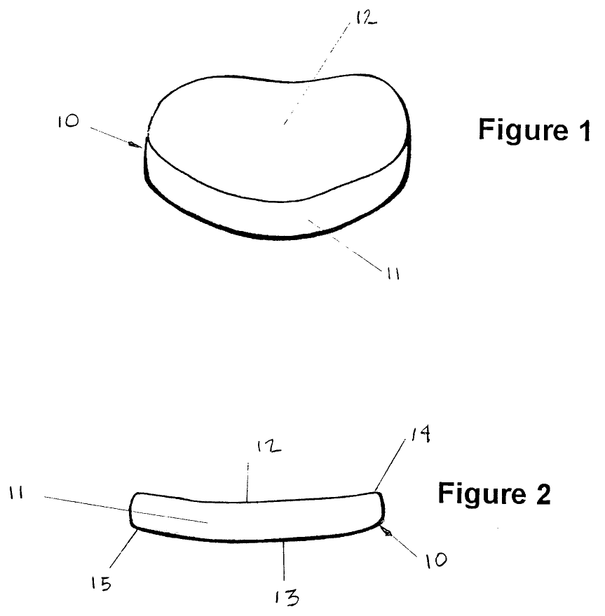

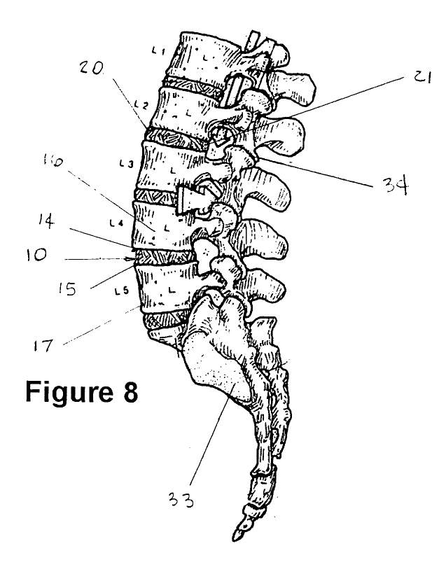

As shown in FIG. 1, the spinal disc body 10 has a circumferential surface 11, a superior, substantially concave surface 12, and an inferior, substantially convex, surface 13. The circumferential surface 11 of spinal disc body 10 corresponds to the annulus fibrosis (“annulus”) of the natural disc. The superior surface 12 and inferior surface 13 of spinal disc body 10 correspond to vertebral end plates (“end plates”) in the natural disc. The interior of spinal disc body 10 corresponds to the nucleus pulposus (“nucleus”) of the natural disc. FIG. 2 demonstrates that the spinal disc body 10 is substantially rectangular when viewed anteriorly. As more fully explained in the description of FIG. 8, the periphery 14 of the superior surface 12 and the periphery 15 of the interior surface 13 are substantially flat in order to provide a good interface with the superior and inferior vertebral bodies, 16 and 17, respectively.

The surfaces of the superior surface 12 and inferior surface 13 are preferably roughened with surface texturing, producing a roughness index of between about 1 nm and about 2 mm in height. The circumferential surface 11 is generally smoother than the roughened superior and inferior surfaces, 12 and 13 respectively.

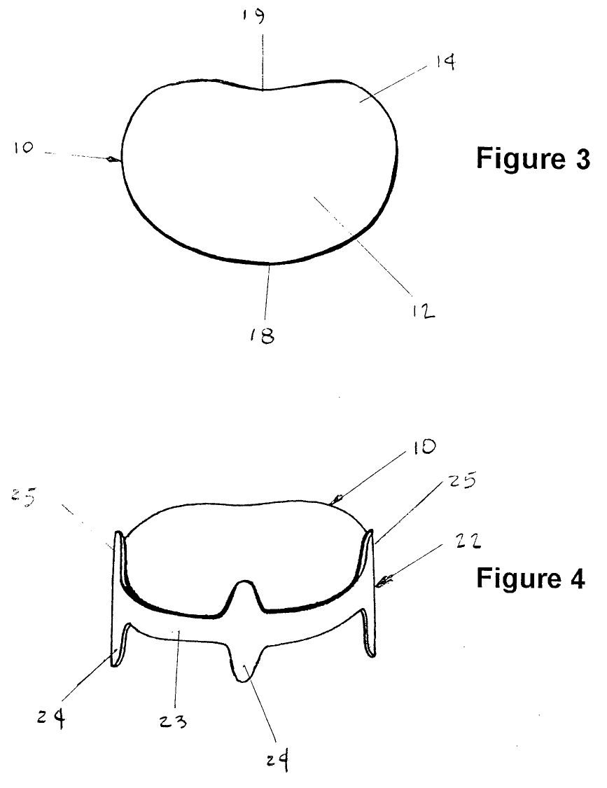

As shown in FIG. 3, the spinal disc body 10 is generally of kidney shape when observed from the superior, or top, view, having an extended oval surface 18 and an indented portion 19.

FIG. 4 depicts the spinal disc body at least partially surrounded by an attachment extension member 22 for attachment to the adjacent vertebral bodies. Attachment extension member 22 includes a band member 23, and a plurality of inferior tabs 24 and superior tabs 25. Band member 23 is adapted to be secured to the extended oval surface 18 of circumferential surface 11. Inferior tabs 24 of the attachment extension number 22 are adapted to be secured to the inferior vertical body 17. And superior tabs 25 of the attachment extension number 22 are adapted to be secured to the superior vertebral body 16.

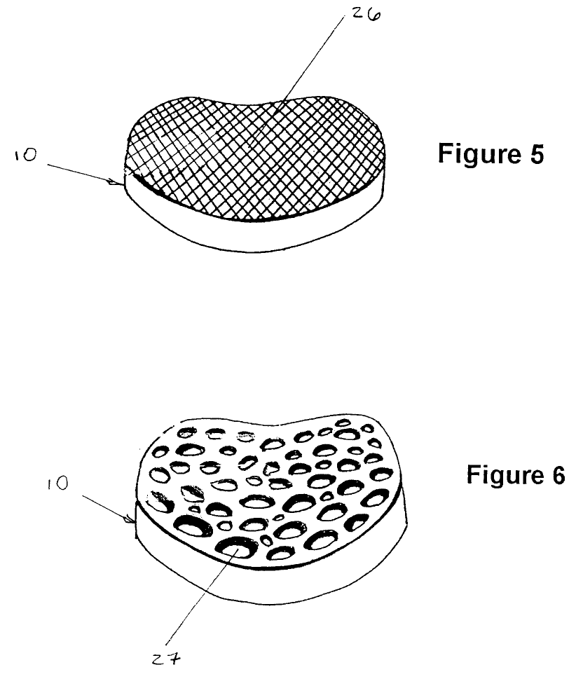

FIG. 5 depicts the spinal disc body 10 in a preferred embodiment wherein the superior surface 12 and inferior surface 13 are covered with fibers or surface treatments such as grooves 26 to enable tissue ingrowth from the adjacent superior vertebral body 16 and inferior vertebral body 17, respectively. In a preferred embodiment, the fibers or surface treatments are applied in a cross-hatched orientation.

FIG. 6 depicts the spinal disc body 10 in a further preferred embodiment wherein the superior surface 12 and the inferior surface 13 are provided with pores or undercuts 27 to enable tissue ingrowth from the adjacent superior vertebral body 16 and the inferior vertebral body 17, respectively. In a preferred embodiment, the pores or undercuts 27 are of varying diameter.

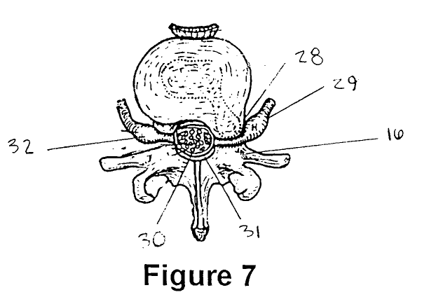

FIG. 7 depicts a degenerated discal area and protruded disc 28 in contact with spinal nerve 29. The cauda equina is shown at 30. The dural sac is shown at 31. And the ganglion is shown at 32. This invention is directed to replacement of the protruded disc 28 with spinal disc body 10 as illustrated in FIG. 8.

FIG. 8 depicts, for example, a spinal disc body 10 implanted between superior vertebral body 16 (L4) and inferior vertebral body 17 (L5). The anterior portion 20 of spinal disc 10 is preferably of greater height than the posterior portion 21 of spinal disc 10 in the sagittal plane. 33 designates the articular surface for the iliac bone, and 34 designates a facet joint.

### EXAMPLE 1

Elastomers useful in the practice of the invention include silicone rubber, polyurethane, polyvinyl alcohol hydrogels, polyvinyl pyrrolidone, poly HEMA, HYPAN™ and Salubria™ biomaterial. Methods for preparation of these polymers and copolymers are well known to the art. The device described in this example is made from an elastomeric cryogel material disclosed in U.S. Pat. Nos. 5,981,826 and 6,231,605, hereby incorporated by reference, that has a mechanical compressive modulus of elasticity of about 1.0 MPa, ultimate stretch of greater than 15%, and ultimate strength of about 5 MPa. The device can support over 1200 N of force.

A preferred hydrogel for use in the practice of this invention is highly hydrolyzed crystalline poly (vinyl alcohol) (PVA). PVA cryogels may be prepared, from commercially available PVA powders, by any of the methods known to the art. Preferably, they are prepared by the method disclosed in U.S. Pat. Nos. 5,981,826 and 6,231,605, the teachings of which are incorporated herein by reference. Typically, 25 to 50% (by weight) PVA powder is mixed with a solvent, such as water. The mixture is then heated at a temperature of about 100 degrees Celsius (C) until a viscous solution is formed. The solution is then poured or injected into a metal or plastic mold such as shown in FIG. 1. The device is allowed to cool to below −10 degree C., preferably to about −20 degree C. The device is frozen and thawed several times until a solid device is formed with the desired mechanical properties. The device can them be partially or completely dehydrated for implantation. The resulting prosthesis has a mechanical elasticity of 2 MPa and has a mechanical ultimate strength in tension and compression of at least 1 MPa, preferably about 10 MPa. The prosthesis made by this method allows for 10 degrees of rotation between the top and bottom faces with torsions greater than 1 N-m without failing. The device thus made does not fracture when subjected to the same load constraints as the natural intervertebral disc. The device is a thus made of a single solid elastomeric material that is biocompatible by cytotoxicity and sensitivity testing specified by ISO (ISO 10993-5 1999: Biological evaluation of medical devices—Part 5: Tests for in vitro (italics) cytotoxicity and ISO 10993-10 2002: Biological Evaluation of medical devices-Part 10: Tests for irritation and delayed-type hypersensitivity.).

### EXAMPLE 2

The prosthetic disc can be made from a variety of elastomers provided the shape, elasticity, biocompatibility, and strength requirements are met. These implantable medical devices can be made from materials such as polyurethane, silicone, hydrogels, collagens, hyalurons, proteins and other synthetic polymers can be used to achieve the desired range of elastomeric mechanical properties. Polymers such as silicone and polyurethane are generally known to have mechanical elasticity values of less than 100 MPa. Hydrogels and collagens can also be made with mechanical elasticity values less than 20 MPa and greater than 1.0 MPa. Silicone, polyurethane and some cryogels typically have ultimate tensile strength greater than 100 or 200 kiloPascals. Materials of this type can typically withstand torsions greater than 0.01 N-m without failing.

The body of the prosthesis may be further reinforced with fibers of polyethylene, polyglycolic acid, poly-paraphenylene terephthalamide, or silk, which are arranged in a circumferential direction, preferably as a complete woven mesh ring within the body of the device, or a crossing structure similar to the natural disc annulus.

The exact size of the prosthetic spinal disc can be varied for different individuals. A typical size of an adult disc is 3 cm in the minor axis, 5 cm in the major axis, and 1.5 cm in thickness, but each of these dimensions can vary by 500% without departing from the spirit of the invention.

### EXAMPLE 3

The device may be fabricated with different percentage weights of PVA at different stages of the molding process to yield a range of mechanical modulus of elasticity within the prosthetic spinal disc such that the elasticity is not constant. Similarly, two elastomers may be combined to yield elasticities that are not constant. Another approach can be to combine fibers or meshes within the device to yield anisotropic elasticity.

### EXAMPLE 4

A form of the device is to have a kidney shape made of a material that will expand to a fixed dimension after placement in the body. A prosthesis was made from a PVA hydrogel described by Peppas, Poly (vinyl alcohol) hydrogels prepared by freezing—thawing cyclic processing. Polymer, v. 33, pp. 3932-3936 (1992); Shauna R. Stauffer and Nikolaos A. Peppas. This prosthesis exhibited swelling characteristics that caused the prosthesis to swell from 5% to six times (600%) its original size over 24 hours when placed in a bath of normal saline. The swelling pressure is measured to be greater than 1 Newton in the cranial-caudal direction of the device. The swelling and expansion can be made from a variety of materials that swell from hydration or osmotic pressure. This swelling and expansion can be used to enhance water transport through the material. The enlargement of the device can also be achieved with the use of mechanical springs that are embedded into the device. Alternatively, the height of the device may be expanded by use of an internal spring made of one or more pieces of metal or plastic that can exert an expansion force greater than 1 Newton. It is anticipated that expansions greater than 10% in height will be useful for this device and are included in this invention.

### EXAMPLE 5

Additional adhesion to the vertebral bodies may be obtained by incorporating surface modifications on the cranial and caudal faces of the prosthesis. The modifications may consist of physical scoring or indentations of the surface, chemical irritants incorporated on the surface, biochemical agents modified on the surface, or small fibers that extend from the faces to stimulate adhesion to a vertebral body or vertebral endplate. These fibers and surface modifications may induce a fibrotic or osteogenic reaction from the person to enhance attachment to the vertebral bodies.

Fibrosis may be induced by a plurality of methods including open pore or rough surfaces, porous structures with undercuts, incorporation of osteoconductive or inductive agents, incorporation of other polymers such as polyester fabric or fibers, incorporation of other biologically active molecules such as tumor necrosis factor or collagen, metal solid or mesh, rough surface with features greater than 5 nanometers (nm). The roughness of the surface may include pores with undercuts of 2 millimeters (mm) in diameter, similar to a sponge. The surface may also be biochemically modified to provide enhanced water transport or physically modified to provide enhanced chemical transport. It is anticipated that there are many ways of modifying the surface characteristics of the prosthesis to achieve the same objective of providing cellular in-growth or attachment by collagen or bone. This invention anticipates these factors and others in this class.

### EXAMPLE 6

The device may have an appendage to allow for immediate fixation in situ. For example, a prosthesis can be made to provide a screw anchor point for fixation in the vertebral body as shown in FIG. 4. Such a device can be made from a cryogel with elasticity between 0.2 and 5 megaPascals with tab extensions. The fixation appendages may extend from the main body of the spinal disc replacement. The elastomer is further surrounded along the circumference of the disc by a material that contains a ring of continuous fiber connected to the fixation appendage labeled as 12.

Attachment may be mechanically achieved by use of fabrics or interposed substances between the expanding body and the vertebrae. The attachments may be biodegradable or permanent. Use of polyester, screws, glues, plates, and other such connectors are anticipated but are not limited to these embodiments.

### EXAMPLE 7

A preferred embodiment is a sterile prosthesis manufactured in a kidney shape for use as a spinal disc prosthesis. The body of the prosthesis is composed of a cryogel material with mechanical compressive modulus between 1.5 MPa and 10 MPa and ultimate tensile stretch greater than 50% in one direction. The material has a swelling characteristic that expands 50% in height when placed in a Normal saline solution. The cranial and caudal surfaces of the prosthesis that contacts the vertebrae have exposed polyester fibers that are embedded into the body and can stimulate a fibrotic reaction for long-term attachment. Further, open cell pores are made to a depth of 2 mm on the cranial and caudal surfaces to provide for boney attachment as shown in FIG. 6. These holes have undercuts to allow for firm attachment between the device and fibrous tissue from the end plates of the vertebral body. A sheet of poly-paraphenylene terephthalamide fabric is molded into the device near the circumferential, cranial and caudal surface and extends for approximately 1 centimeter beyond the body of the device. The fabric appendages are used to attach the device to the sides of the vertebrae.

While several examples of the present invention have been described, it is obvious that many changes and modifications may be made thereunto without departing from the spirit and scope of the invention.

1. An implantable prosthesis of shape generally similar to that of a spinal intervertebral disc, comprised of a biocompatible elastomer with a mechanical elasticity less than about 100 megaPascals, with an ultimate strength in tension generally greater than about 100 kiloPascals, that exhibits the flexibility to allow at least 2 degrees of rotation between the top and bottom faces with torsions greater than 0.01 N-m without failing.

2. A prosthesis according to claim 1 wherein the device has ultimate strength to withstand a compressive load greater than 1 MegaPascals.

3. A prosthesis according to claim 1 wherein the material used for the device has a mechanical ultimate strength greater than 5 MPa.

4. A prosthesis according to claim 1 wherein the device is made of a single solid elastomeric material.

5. A prosthesis according to claim 1 wherein the elastomer has a mechanical elasticity greater than 1.0 MPa.

6. A prosthesis according to claim 1 wherein the elastomer has a mechanical elasticity less than 20 MPa.

7. A prosthesis according to claim 1 wherein device has a mechanical elasticity less than 10 MPa and greater than 200 KPa.

8. A prosthesis according to claim 1 wherein elastomer has a mechanical elasticity that is not constant.

9. A prosthesis according to claim 1 wherein the delivered size of the prosthesis can expand at least 5% in at least one dimension over one day, in saline.

10. A prosthesis according to claim 1 wherein the delivered size of the prosthesis can expand at least 50% in at least one dimension in vivo without injection of material.

11. A prosthesis according to claim 1 wherein the delivered size of the prosthesis can expand at least 20% over one day in at least one dimension in vivo and can generate a cranial-caudal force of greater than 1 Newton.

12. A prosthesis according to claim 1 wherein the delivered size of the prosthesis can expand at least 100% by a combination of springs and elastomeric components.

13. A prosthesis according to claim 1 that is further modified to provide specific surface characteristics.

14. A prosthesis according to claim 13 wherein the surface characteristics are physically or biochemically modified to provide enhanced adhesion to a vertebral body.

15. A prosthesis according to claim 13 wherein the surface includes, in part, a fabric.

16. A prosthesis according to claim 13 wherein the surface includes, in part, a metal solid or mesh.

17. A prosthesis according to claim 13 wherein the surface includes, in part, a porous structure with undercuts.

18. A prosthesis according to claim 13 wherein the surface includes, in part, a rough surface greater than 5 nanometers.

19. A prosthesis according to claim 13 wherein the surface includes, in part, a bioactive molecule.

20. A prosthesis according to claim 1 wherein the surface characteristics of the prosthesis are modified to provide cellular ingrowth.

21. A prosthesis according to claim 1 wherein the surface characteristics are biochemically modified to provide enhanced water transport.

22. A prosthesis according to claim 1 wherein the surface characteristics are physically modified to provide enhanced chemical transport.

23. A prosthesis according to claim 1 wherein the device is made of a single elastomer with elasticity between 0.2 and 5 megaPascals with tab extensions for fixation to the adjacent vertebral bodies.

24. A prosthesis according to claim 1 wherein the disc is composed of a material that contains a ring of continuous fiber.

25. A prosthesis according to claim 1 that contains appendages to allow for physical attachment to the vertebral body and to prevent dislodgement of part in situ.

26. A prosthesis according to claim 1 wherein the material is a cryogel.

27. A prosthesis according to claim 1 wherein the material is a composite material composed of more than one substance.

28. A prosthesis according to claim 1 that is a permanent implantable medical device.

29. A sterile prosthesis according to claim 1 wherein the body is manufactured as an oval or kidney shape for use as a spinal disc prosthesis that expands 20% in height when placed in normal saline solutions, has exposed fibers on the cranial and caudal surfaces, has a body composed of a biocompatible elastomer compressive modulus between 1.5 MPa and 10 MPa, ultimate compressive strength greater than 1 MPa, ultimate tensile stretch greater than 25% in one direction, and contains fabric extensions from the body for attachment to the sides of the vertebrae.

30. Use of the prosthesis of claim 1 as a medical implant for the spinal disc.

31. Use of the prosthesis of claim 1 that is inserted by surgery into the intervertebral space.

32. Use of the prosthesis of claim 1 for separation of two boney surfaces.

33. Use of the prosthesis of claim 1 for veterinary applications.

34. An implantable spinal disc body having a superior surface and an inferior surface joined by a circumferential surface comprised of a biocompatible elastomer with a mechanical elasticity less than about 100 megaPascals and an ultimate strength in tension greater than about 100 kiloPascals.

35. The implantable spinal disc body of claim 34 wherein the implantable spinal disc superior and inferior surfaces are of a kidney shaped and formed by an extended oval surface and an indented surface, and wherein the cross-section of the implantable spinal disc is substantially rectangular.

36. The implantable spinal disc body of claim 34, wherein the periphery of the superior and inferior surfaces is substantially flat.

37. The implantable spinal disc body of claim 34, wherein the superior and inferior surfaces have a roughness index of between about 1 nm and about 2 mm in height.

38. The implantable spinal disc body of claim 37, wherein the circumferential surface has a roughness index of less than 1 mm.

39. The implantable spinal disc body of claim 34, wherein the implantable spinal disc body is at least partially surrounded by an attachment extension member having a plurality of superior and inferior tabs connected to a band member for attachment of the implantable spinal disc to adjacent superior and inferior vertebral surfaces, respectively.

40. The implantable spinal disc body of claim 34, wherein the superior and inferior surfaces are covered with a surface treatment to promote attachment to the adjacent vertebral bodies.

41. The implantable spinal disc body of claim 34, wherein the superior and inferior surfaces are provided with a plurality of pores to promote tissue ingrowth.

42. The implantable spinal disc body of claim 34 wherein the anterior portion of the implantable spinal disc body is of greater thickness than the posterior portion.

43. An implantable spinal disc body of biocompatible elastomer material having a mechanical elasticity less than about 100 megaPascals and an ultimate strength in tension greater than about 100 kiloPascals, comprising:
a substantially concave superior surface having a substantially flat periphery surface;
a substantially convex inferior surface having substantially flat periphery;
the superior and inferior surfaces being joined by a circumferential surface; and
the implantable spinal disc body being further characterized as being of a kidney shape formed by an extended oval surface and an indented portion, having a substantially rectangular cross-section, and having an anterior portion of greater thickness than the posterior portion.

44. The implantable spinal disc body of claim 43 wherein the superior and inferior surfaces have a roughness index of between about 1 nm and about 2 mm in height and the circumferential surface has a roughness index of less than 1 mm.

45. The implantable spinal disc body of claim 43 further comprising:
an attachment extension band member at least partially surrounding the circumferential surface of the implantable spinal disc body; and
a plurality of superior and inferior tabs extending from said attachment extension band member for attachment of the implantable spinal disc body to adjacent superior and inferior vertebral surfaces, respectively.
